(19) United States
(12) Patent Application Publication
(10) Pub. No.: US 2011/0311593 A1
(43) Pub. Date: Dec. 22, 2011

(54) N-HYDROXYLATED AMIDINE-, GUANIDINE- AND/OR AMINOHYDRAZONE-TYPE PRODRUGS FOR APPLICATION ON THE SKIN

(76) Inventors: Rudolf Matusch, Marburg (DE); Hans-Rainer Hoffmann, Neuwied (DE); Bodo Asmussen, Bendorf (DE); Andreas Koch, Melsbach (DE)

(21) Appl. No.: 13/148,422

(22) PCT Filed: Feb. 5, 2010
(86) PCT No.: PCT/EP10/00715

§ 371 Date: Aug. 8, 2011

(30) Foreign Application Priority Data
Feb. 10, 2009 (DE) 10 2009 008 256.5

(51) Int. Cl. A61K 9/00; C07D 401/14; C07D 413/12; C07D 205/04; A61P 17/00; C07H 15/238; A61K 31/397; A61K 31/4439; A61K 31/7056; A61K 31/7052; C07D 413/14; C07D 403/12

(52) U.S. Cl. 424/400; 546/269.1; 546/273.4; 548/133; 548/953; 544/324; 544/323; 544/405; 540/480; 536/16; 514/210.17; 514/210.18; 514/37

(57) ABSTRACT

The invention relates to N-hydroxylated amidines, guanidines and aminohydrazones for application on the skin. In particular, the invention relates to transdermal therapeutic systems containing N-hydroxylated amidines, guanidines and aminohydrazones as a prodrug, and to methods for producing and using such systems.

The invention relates to N-hydroxylated amidines, guanidines and aminohydrazones for application via the skin. In particular, the invention relates to transdermal therapeutic systems which comprise N-hydroxylated amidines, guanidines and aminohydrazones as prodrug, and to methods for producing such systems.

Medicaments of the amidine type (e.g. pentamidine as gold standard for the treatment of Pneumocystis carinii pneumonia in Aids patients), of the guanidine type (e.g. debrisoquine) and of the aminohydrazone type (e.g. guanabenz for the treatment of high blood pressure) have, on account of the structure of their nitrogen-containing functional groups, a high basicity (pkₐ=11-12) and are consequently predominantly in protonated (ionized) form at physiological pH values (e.g. in the stomach at pH 1 and in the intestine at pH 6.8 and even in the blood at pH 7.4). This in turn means that the passage, required for absorption, of lipophilic membranes by passive diffusion, such as, for example, the gastrointestinal passage or the blood brain barrier, can only be overcome with very great difficulty. The bioavailability and also the pharmacological effect of such medicaments resulting therefrom is generally very poor or low. So too for the application of such active ingredients by means of transdermal therapeutic systems, so-called TTS.

The use of a number of amidine prodrugs of pentamidine are described in EP-A-0 708 640 and U.S. Pat. No. 5,786,383, but only in the use for the oral route.

N-hydroxylated derivatives for guanidines such as e.g. N-hydroxydebrisoquine or aminohydrazones such as e.g. guanoxabenz are likewise already known without there being any references to the use as prodrugs for a transdermal application.

WO 97/23499 describes amidoximes of certain thrombin inhibitors. Oral and parenteral applications are mentioned in general terms as well as other types of application.

Transdermal therapeutic systems (TTS) have been known in the specialist field and marketed for a number of years. Transdermal therapeutic systems are self-adhesive pharmaceutical preparations with a fixed application area to be applied to the skin which release a medicament to the human or animal body in a manner controlled according to time and amount.

The therapeutic progress of these systems compared with traditional application forms is that the active ingredient is not passed to the body in a stop-start manner, as for example when taking tablets, but continuously.

As a result of this, on the one hand, the duration of effect of a medicament is extended, and secondly side-effects are largely prevented by avoiding unnecessary blood-level peaks.

For such systems, coated, flat forms using various polymers, e.g. polyethylene terephthalate, polyisobutylene or polysiloxane, are usually used.

However, the aforementioned active ingredients of the amidine, guanidine and aminohydrazone type unfortunately rule themselves out for an application as TTS on account of their high basicity since they would invariably lead to massive and severe skin irritations (comparable with caustic burns caused by sodium hydroxide solution) and thus preclude themselves from acceptance by the patient.

On account of the aforementioned advantages of the TTS application, however, there was a need to make these substance classes accessible to a TTS application. Accordingly, it was an object of the present invention to supply amidines, guanidines and aminohydrazones in a suitable manner for transdermal application.

This object is achieved by a system for the transdermal application of active ingredients, comprising at least one compound which has at least one derivatized amidine, guanidine or aminohydrazone group of the following general formula 1a-c:

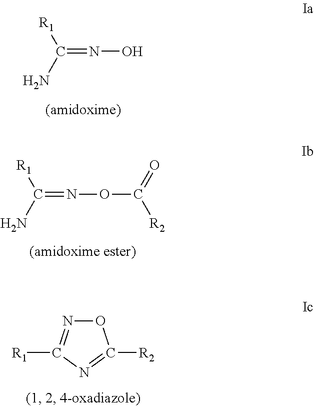

where
R₁ is a radical which is selected from the group consisting of:
   straight-chain or branched C₁-C₂₀-alkyl, C₅-C₁₄-aryl,
   straight-chain or branched C₁-C₂₀-alkyl-NH—,

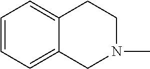

or
R₃—C═N—NH—, where R₃ is C₁-C₂₀-alkyl or C₅-C₁₄-aryl,
where these radicals R₁ are unsubstituted or substituted with:
   straight chain, branched or cyclic C₁-C₈-alkyl, —OH, —NH₂, —NO₂, —CN, —C(O)OH, —C(O)O—C₁-C₄-alkyl, —Cl, —Br, —I, and where
one or more CH₂ groups in these radicals R₁ can be replaced by —O—, —S—, —NH—, —N(C₁-C₄-alkyl)- or —C(O)— and where
two H atoms of one or two methylene groups and/or one or two NH groups can be replaced by a C₂-C₄-alkylene group and where
R₂ is a C₁-C₂₀-alkyl or C₅-C₁₄-aryl radical.

The details in brackets refer to the compound type which is formed during the derivatization.

Preferably, R₁ is a radical of the formula II

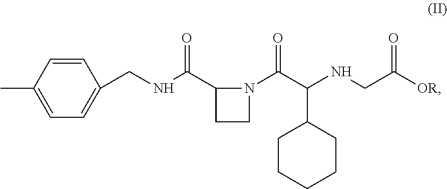

in which
R is —R₄ or -A₁C(O)N(R₅)R₆ or -A₁C(O)OR₅;
A₁ is C₁-C₅-alkylene;
R₄ is H, C₁-C₁₀-alkyl or C₁-C₃-alkylphenyl (where the last-mentioned group is unsubstituted or is substituted with C₁-C₆-alkyl, C₁-C₆-alkoxy, —NO₂ or Cl, Br or I);
R₅ and R₆ independently of one another, are H, C₁-C₆-alkyl, phenyl or 2-naphthyl or, if R is -A₁C(O)N(R₅)R₆, are together with the nitrogen to which they are bonded, pyrrolidinyl or piperidinyl.

Particular preference is given to compounds of the formula I, in which R is: H, Et-, nPr—, tBu-, Prl-C(O)CH₂CH₂CH₂—, Ch-NHC(O)CH₂—, (nPr)₂NC(O)CH₂—, cyclooctyl-, tBuCH₂—, (2-Me)Bn-, ChCH₂—, Ch-, PhC(Me)₂-, (Me)₂CHC(Me)₂-, Bn-, iPr—, MeO(O)C—C(═CHEt)CH₂— or Men-.

Here, Bn=benzyl, Bu=butyl, Ch=cyclohexyl, Et=ethyl, Me=methyl, Men=(1R,2S,5R)-menthyl, Pr=propyl, Prl=N-pyrrolidinyl. The prefixes n, s, i and t have their usual meaning: normal, iso, secondary and tertiary.

If not already encompassed by the above definition of the radical R₁, the following compounds also fall under the corresponding non-derivatized amidines, guanidines and aminohydrazones:

### Group of Amidines

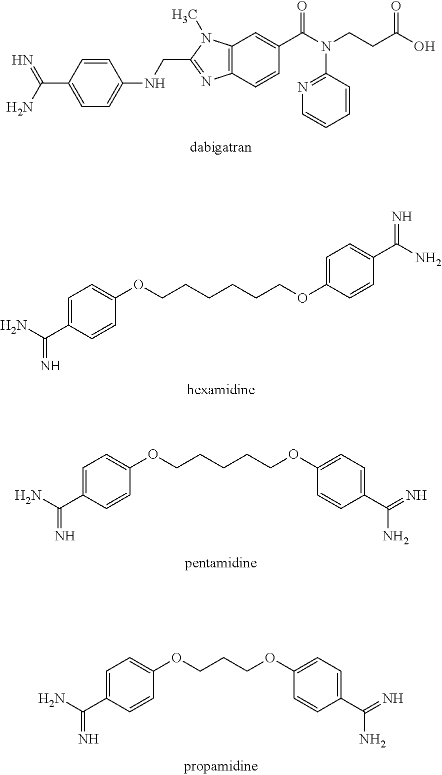

### Group of Guanidines

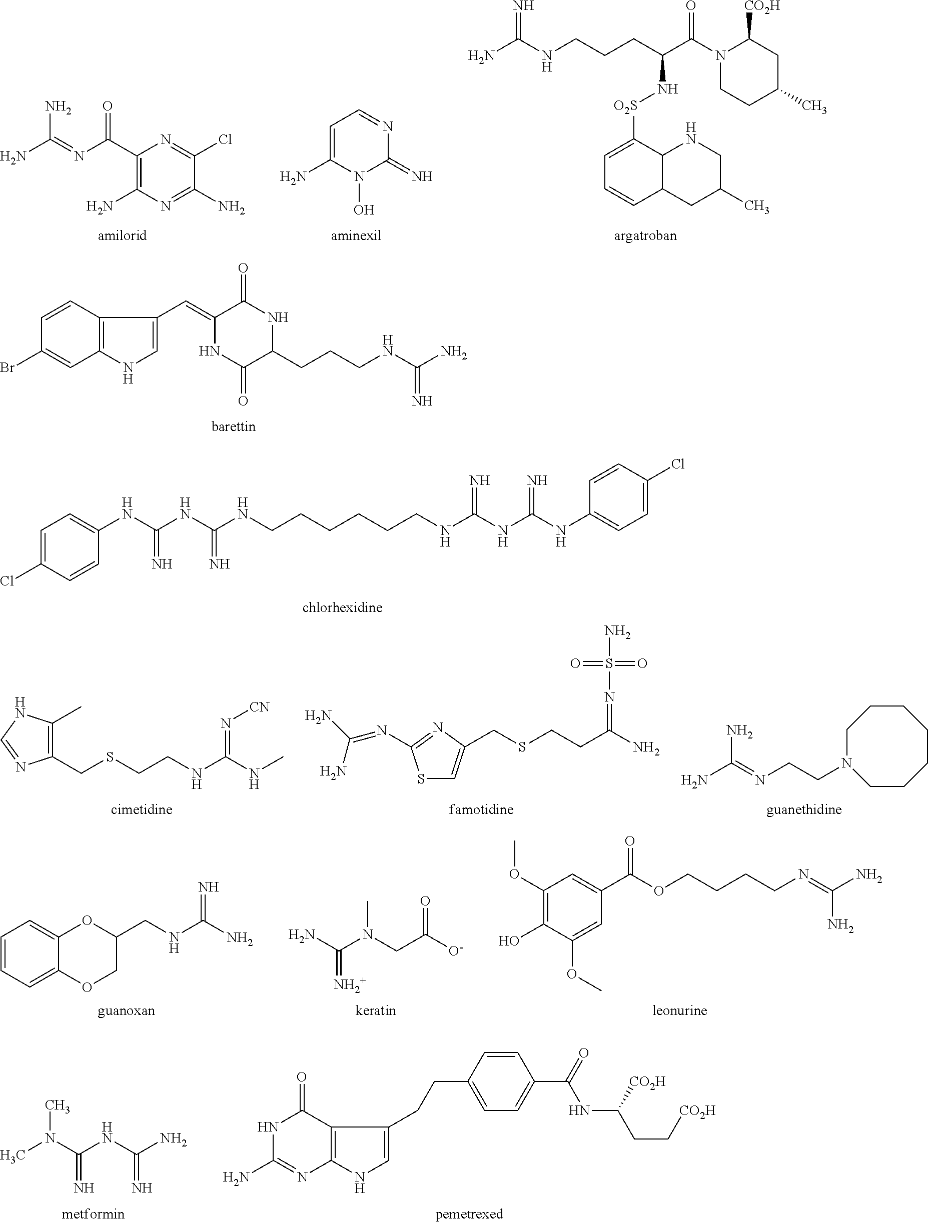

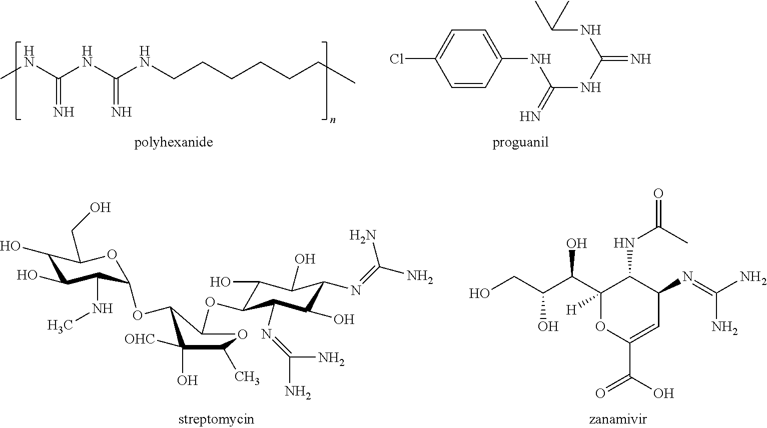

### Group of Aminohydrazones

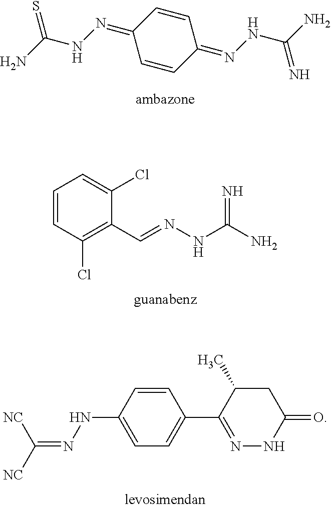

As a result of the N-hydroxylation and possible further derivatization of the N-hydroxyl group to the corresponding amidoxime esters or 1,2,4-oxadiazoles, the pkₐ values can be lowered below 5. Consequently, at physiological pH values, these compounds are now present predominantly in unprotonated form and are thus lipophilic, meaning that lipid membranes can be passed again without problem. As a result, the bioavailability and therefore the degree of pharmacological effect increases at the same time. On account of their lowered basicity, these N-hydroxylated forms (amidoximes) are also again suitable for an application as TTS, especially since the skin accessibility is also surprisingly increased at the same time as a result of the increased lipophilicity of the amidoximes (see example 1).

Since the process of N-hydroxylation is reversible by endogenous enzymes such as esterases and N-reductases such as, for example, cytochrome P 450, cytochrome b5, NADH cytochrome b5 reductase or also NADH on its own, the N-hydroxyl derivatives of the aforementioned substance groups represent suitable prodrugs which can be converted again to the effective form in the body.

The transdermal therapeutic system (TTS) preferably has a back layer facing outwards (=the side facing away from the skin) which limits the diffusion of water, and, on the side facing the skin, a base material which comprises the prodrug-containing formulation.

Essential constituents of the base material to be mentioned by way of example are polymers such as rubber, rubber-like synthetic homopolymers, copolymers or block polymers, polyacrylic acid esters and copolymers thereof, polyurethane copolymers of ethylene, polyisobutylene, polybutylene and polysiloxanes. In principle, all polymers are suitable which are essentially water-insoluble and do not exert any disadvantageous effects on the person when in direct and indirect contact with the skin.

Since the adhesive bond can also take place via an additionally applied adhesive layer, the base material does not necessarily have to be made primarily pressure-sensitive-adhesive, although this property is preferred for a particularly thin and flexible, non-applying system structure, which would also make a single-layer system possible.

Further substances known to the person skilled in the art having a functional influence on the base material can be used, such as e.g. plasticizers, tackifiers, absorption promoters, stabilizers or fillers.

Suitable auxiliaries which can be used for the prodrug-containing formulation are water-soluble or water-swellable polymers. Among these, mention may be made by way of example of: polyvinyl alcohol and its copolymers, polyvinylpyrrolidone and its copolymers, polyethylene glycols, preferably with a molecular weight of above 1000 daltons (which are thus solid at room temperature). The above polymers can advantageously consist of partial crosslinked structures for the controlled dispersion of the prodrugs in the base material. Further polymers which can readily be used are alginates, pullulan, guar gum with gum arabic or other vegetable gums, cellulose, in particular microcrystalline cellulose and its derivatives such as e.g. methylcellulose, hydroxyethylcellulose, hydroxymethylpropylcellulose etc., but also other carbohydrates such as e.g. starch, particularly preferably in derivatized or modified form. However, peptidic polymers such as collagen and gelatin are also certainly suitable. Water-soluble and water-swellable polymers have the advantage that, in the event of the absorption of water, they do not suddenly become ductile and diffusible, but only do so gradually and consequently release the enclosed prodrug(s) more evenly. This is particularly useful in application cases in which the prodrugs are only intended to be included in the release process stepwise.

Should a more rapid conversion be preferred, small, molecular, water-soluble substances can be used advantageously as the sole or admixed auxiliaries for formulating the prodrug-containing formulation. Of primary suitability here are, on account of their property of forming glass-like solidifying, diffusion-resistant particles, sugars and their derivatives, predominantly sucrose, glucose, lactose, fructose, but also sugar alcohols, such as sorbitol or mannitol. In principle, all pharmaceutically compatible water-soluble substances which have the property of liquefying under a water-vapor stress of about 98 percent relative humidity (as is provided by the skin), such as e.g. sodium chloride, urea, malic acid, citric acid, are also suitable.

Additives for achieving further functionalities known to the person skilled in the art, such as e.g. stabilizers (in particular antioxidants), fillers, but also micellar-acting modifiers (lecithins) can be provided according to the particular requirement.

Besides the prodrug-containing base material essential to the invention, which, in the simplest case, together with a back layer, can already form a complete TTS system, further system constituents which are known to the specialist world can be usefully combined with the inventive principle.

Consequently, the TTS according to the invention, preferably in the form of a transdermal plaster, can in principle be constructed like systems known from the prior art.

Among the aforementioned further system constituents, mention is to be made e.g. of polymer-containing layers and also membranes which can have a property controlling the prodrug feed to the skin, or else can moderate the all too rapid absorption of moisture from the skin.

As materials for such membranes, polyethylene, polyamide, ethylene vinyl acetate copolymers, but also porous layers filled with low molecular weight substances are customary and known to the person skilled in the art. With or without the use of a membrane, additional adhesive layers can also be applied for better fixing on the skin side; the essential auxiliaries of these have already been specified above in the explanation of the base materials. Here, highly diffusible lipophilic polymers are to be mentioned particularly preferably, such as e.g. polysiloxanes and acrylate copolymers. The principle according to the invention can moreover be combined with further methods of increasing absorption. For example, penetration promoters may be added which increase the permeability of the skin, and physical principles, such as iontophoresis, electroporation and also ultrasound, and also microneedles can be used.

The back layer of transdermal systems for the use according to the invention can consist e.g. in a water-vapor-blocking/occlusively acting polyester (polyethylene terephthalate) membrane which protects both against prodrug loss and also against moisture loss. The water vapor loss can be moderated through appropriate adaptation of the thickness or choice of different materials (polyethylene, polyurethane, or laminates of different thermoplastic raw materials).

Producing the systems according to the invention themselves is possible in myriad ways. The following possibilities are stressed in particular and preferred, but ultimately exemplary and relate in particular to producing the prodrug reservoir according to the invention (base material with prodrug-containing formulation).

Otherwise, the construction/production of the TTS according to the invention (layer structure, materials, auxiliaries and additives) can take place as described by the methods known to the person skilled in the art from the prior art (see e.g. “Dermatological Formulation and Transdermal Systems”, Kenneth A. Walters and Keith R. Brain in Dermatological and Transdermal Formulations, NY 2002, Marcel Dekker, pages 319-399).

Thus, for example, the active ingredient precursor (prodrug) can be supplied to a solution or suspension of the base material which is present in organic solution or even produced without solvents (hot-melt rapid method), whereupon, following subsequent coating on the back layer and drying of the layer, a product that is capable of functioning straight after punching is obtained. The prodrug reservoir (base material and prodrug formulation) is designed here to be self-adhesive.

The precise choice of the dimensions layer thicknesses and polarities of the individual system components must naturally be determined separately for each individual application case.

The invention is illustrated in more detail below by reference to examples.

### EXAMPLE 1

### Preparation of Benzamidoxime

Benzamidoxime is prepared in accordance with Tiemann and Krüger (Tiemann, F., Chem. Berichte 17, 126 (1884)) by the addition reaction of hydroxylamine onto the corresponding nitrile (phenylacetonitrile).

0.01 mol of phenylacetonitrile (1.172 g) is dissolved in 100 ml of ethanol. Then, with brief stirring until complete dissolution, 0.01 mol of hydroxylamine hydrochloride (0.7 g) and 0.01 mol of NaHCO₃ (0.84 g), predissolved in 50 ml of water, are introduced into this clear solution. This solution is then held under reflux for 18 hours at a temperature between 60 and 80° C.

After cooling, the residue is filtered off and recrystallized from hot water.

Melting point: 79-80° C.

### EXAMPLE 2

### Comparison In Vitro

### Skin Permeation Profiles of Amanidine Prodrugs

System: in-vitro permeation cell (9 ml acceptor volume/1.539 cm² diffusion area, 32° C.)
Acceptor medium: phosphate buffer pH 5.5+0.1% NaN₃ according to DAB 10
Active ingredient: benzamidine, benzamidoxime (from Ex. 1)
Skin type: human full skin, ID 899 (belly female, age 48 years)
Vehicle: suspension in olive oil (2% by weight, 50 mg suspension per cell), applied to a circular nonwoven disk made of synthetic wool (Paratex type I/20) and occlusively covered with a PSA film (silicone adhesive) as “over-plaster”.

| | Active | | | | | |
|---|---|---|---|---|---|---|
| No | ingredient | 0 h | 8 h | 24 h | 32 h | 48 h |
| 1 | Benzamidine | 0 | 2.24 | 13.7 | 21.2 | 41.3 |
| 2 | Benzamidinoxime | 0 | 50.2 | 235 | 315 | 403 |

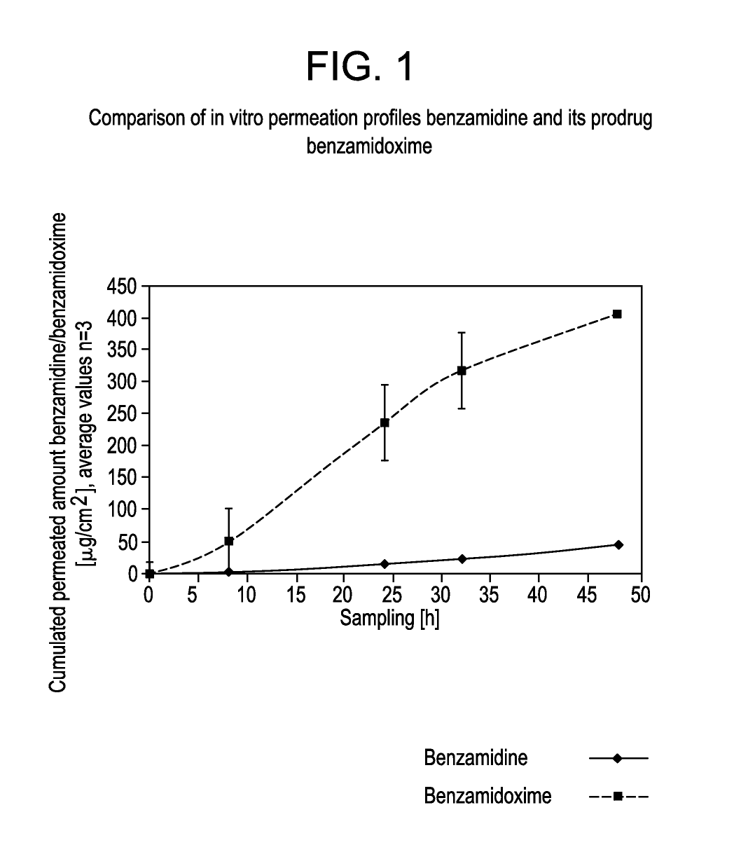

FIG. 1 shows the comparison of the permeated amounts of benzamidine and benzamidoxime, with the advantage of the benzamidoxime being clearly visible.

1. A system for the transdermal application of active ingredients comprising at least one compound as a prodrug which has at least one derivatized amidine group or one guanidine group or one aminohydrazone group of the following general formula Ia-c: 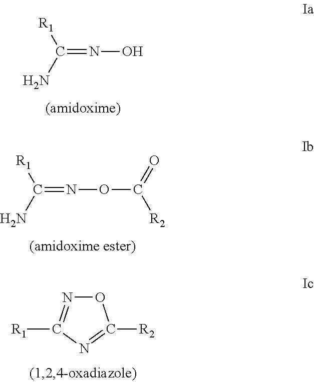
where
R₁ is a radical which is selected from the group consisting of:
straight-chain or branched C₁-C₂₀-alkyl, C₅-C₁₄-aryl,
straight-chain or branched C₁-C₂₀-alkyl-NH—, 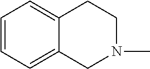
or
R₃—C═N—NH—, where R₃ is C₁-C₂₀-alkyl or C₅-C₁₄-aryl,
where these radicals R₁ are unsubstituted or substituted with:
straight-chain, branched or cyclic C₁-C₈-alkyl, —OH, —NH₂, —NO₂, —CN, —C(O)OH, —C(O)O—C₁-C₄-alkyl, —Cl, —Br, —I, and where
one or more CH₂ groups in these radicals R₁ can be replaced by —O—, —S—, —NH—, —N(C₁-C₄-alkyl)- or —C(O)— and where
two H atoms of one or two methylene groups and/or one or two NH groups can be replaced by a C₂-C₄-alkylene group and where
R₂ is a C₁-C₂₀-alkyl or C₅-C₁₄-aryl radical.

2. The system as claimed in claim 1, where R₁ is a radical of the formula II 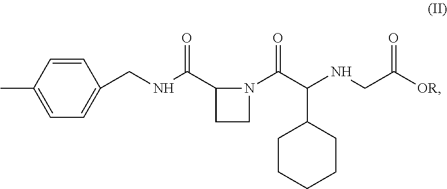
in which
R is —R₄ or -A₁C(O)N(R₅)R₆ or -A₁C(O)OR₅;
A₁ is C₁-C₅-alkylene;
R₄ is H, C₁-C₁₀-alkyl or C₁-C₃-alkylphenyl, where the last-mentioned group is unsubstituted or is substituted with C₁-C₆-alkyl, C₁-C₆-alkoxy, —NO₂ or Cl, Br or I;
R₅ and R₆ independently of one another, are H, C₁-C₆-alkyl, phenyl or 2-naphthyl or, if R is -A₁C(O)N(R₅)R₆, are together with the nitrogen to which they are bonded, pyrrolidinyl or piperidinyl.

3. The system as claimed in claim 2, where R is: H, Et-, nPr—, tBu-, Prl-C(O)CH₂CH₂CH₂—, Ch-NHC(O)CH₂—, (nPr)₂NC(O)CH₂—, cyclooctyl-, tBuCH₂—, (2-Me)Bn-, ChCH₂—, Ch-, PhC(Me)₂-, (Me)₂CHC(Me)₂-, Bn-, iPr—, MeO(O)C—C(═CHEt)CH₂— or Men-.

4. A system for the transdermal application of active ingredients comprising at least one compound as a prodrug, which has at least one derivatized amidine group or one guanidine group or one aminohydrazone group of the following general formula IIIa-c: 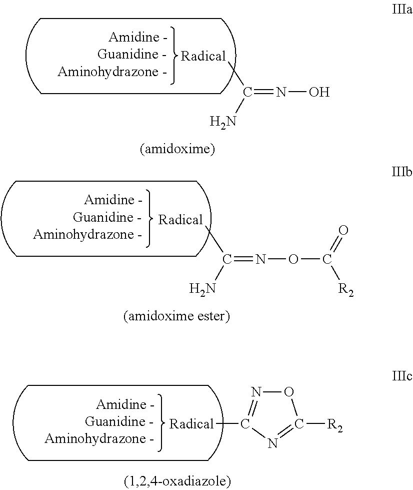
where
R₂ is a C₁-C₂₀-alkyl or C₅-C₁₄-aryl radical, and
where the amidine radical, guanidine radical, aminohydrazone radical is selected from one of the following non-derivatized amidines, guanidines and aminohydrazones:

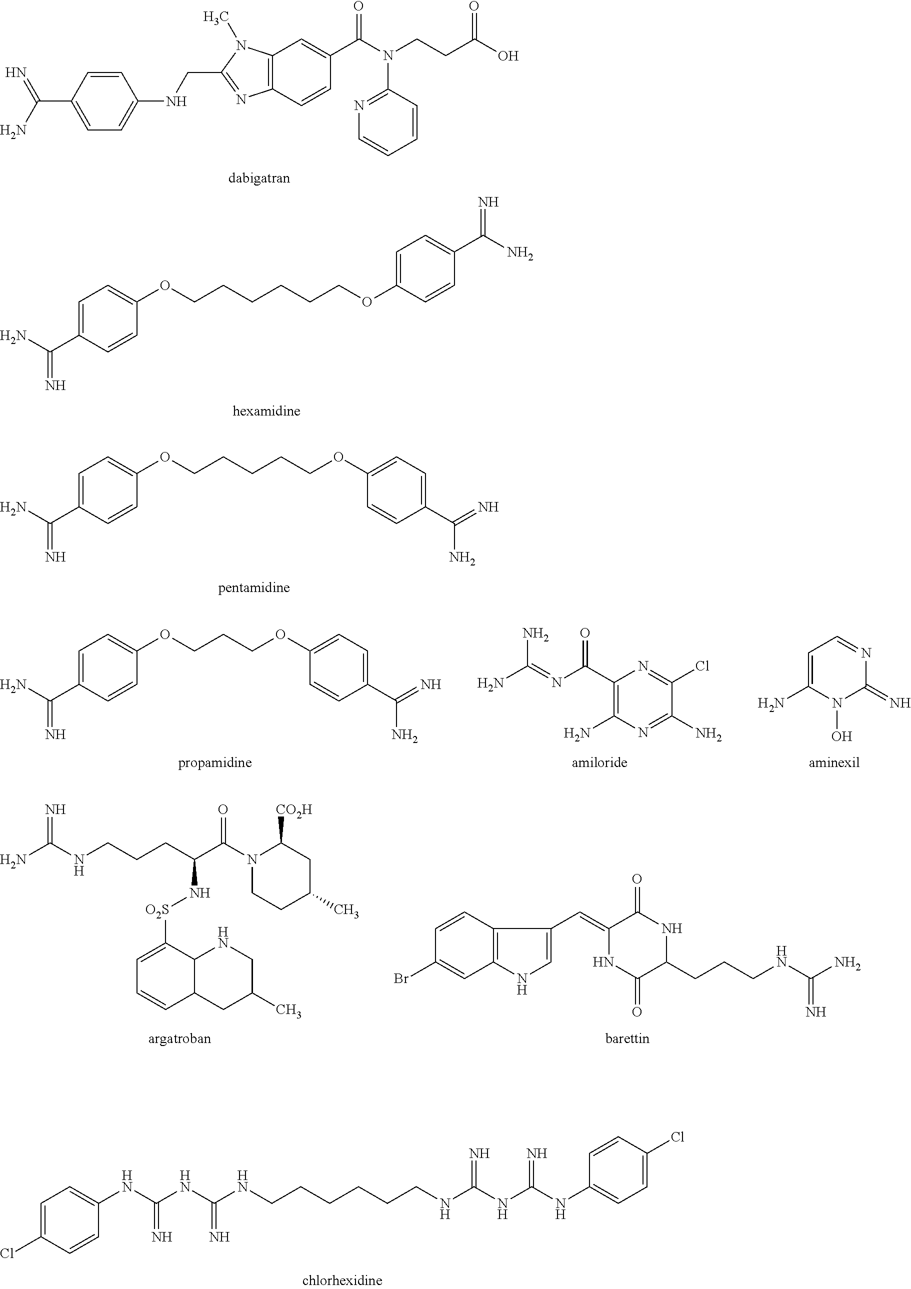

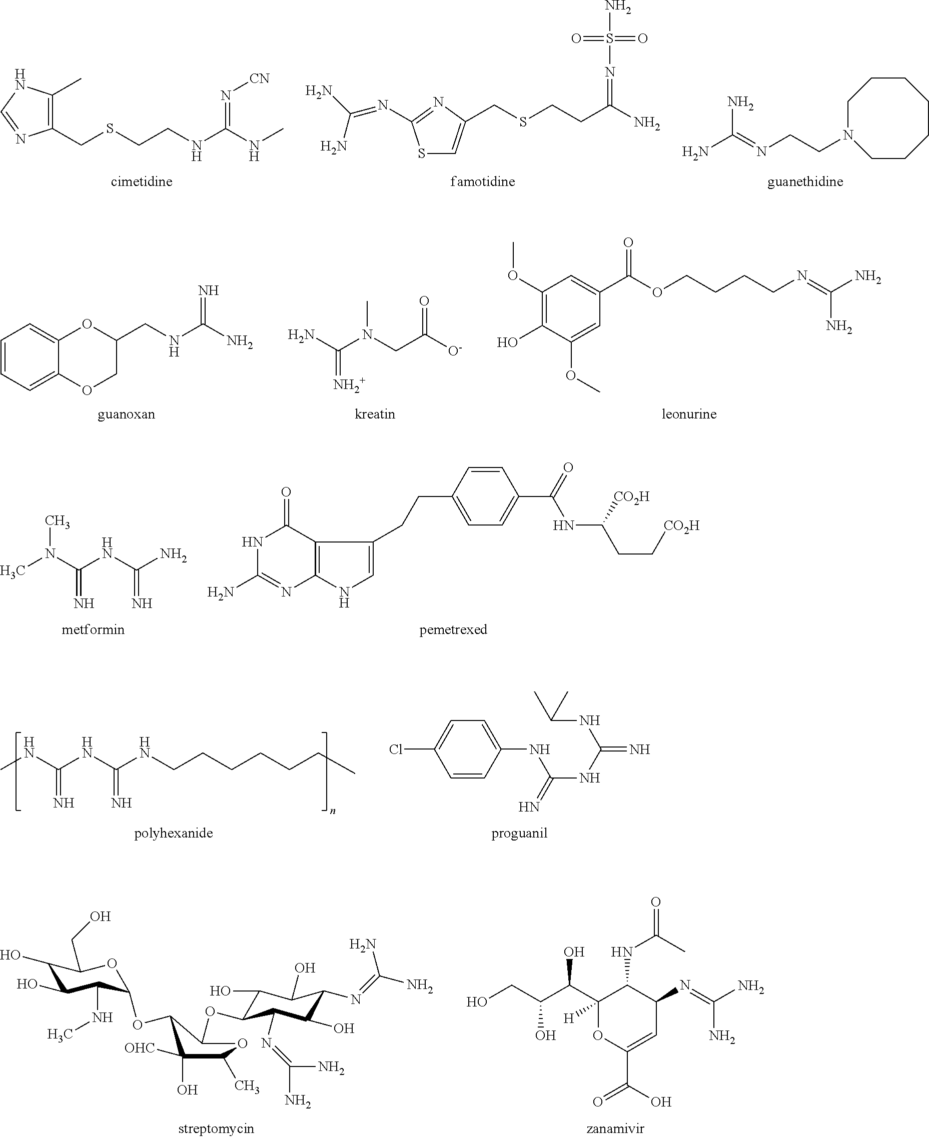

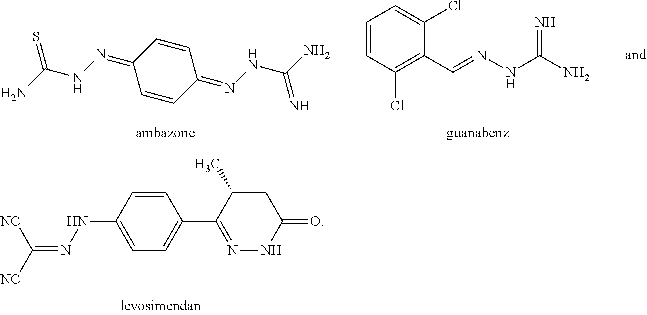

5. The system as claimed in claim 1, wherein said system has a layer structure and comprises at least two layers, of which one is a layer containing prodrug.

6. The system as claimed in claim 5, wherein said system comprises two layers, a back layer facing outwards and a layer containing the prodrug.

7. The system as claimed in claim 5, wherein said system has an adhesive layer.

8. The system as claimed in claim 5, wherein said system has a control membrane.

9. The system as claimed in claim 1, wherein said system is a transdermal therapeutic system.

10. The system as claimed in claim 5, wherein the prodrug-containing layer comprises rubber, rubber-like synthetic homopolymers, copolymers or block polymers, polyacrylic acid esters or copolymers thereof, polyurethanes, polyisobutylene, polybutylene or polysiloxanes.

11. The system as claimed in claim 5, wherein the prodrug-containing layer comprises further auxiliaries or additives.

12. A method for producing the system as claimed in claim 1, comprising introducing the prodrug into a solution or suspension of a base material, said base material comprising rubber, rubber-like synthetic homopolymers, copolymers or block polymers, polyacrylic acid esters or copolymers thereof, polyurethanes, polyisobutylene, polybutylene or polysiloxanes.

13. The method as claimed in claim 12, where the introducing step comprises introducing the prodrug into solvent free base material.

14. A method of releasing medicament comprising applying a system as claimed in claim 5 to a patient's skin.
